(19) United States
(12) Patent Application Publication
(10) Pub. No.: US 2005/0158277 A1
(43) Pub. Date: Jul. 21, 2005

(54) Method to genetate non virulent microorganisms from pathogenic ones through permanent genetic modification of their biological membrane for vaccine production

(76) Inventors: Roberto Cinquegrani, Napoli (IT); Sergio Colonna Romano, Napoli (IT)

Correspondence Address: Jay S Cinamon;Abelman, Frayne & Schwab, 150 East 42nd Street, New York, NY, 10017, US

(21) Appl. No.: 10/479,653

(22) PCT Filed: May 29, 2002
(86) PCT No.: PCT/EP02/05893

(30) Foreign Application Priority Data
May 30, 2001 (IT) RM01A000295

(51) Int. Cl. A61K 48/00; C12N 15/74

(52) U.S. Cl. 424/93.2; 435/471

(57) ABSTRACT

Here we describe a methodology to generate non virulent microorganisms from pathogenic ones through permanent genetic modification of the physical state of their membrane (MPS). Thus, at the onset of infection, in these transformed organisms, as they infect a host (e.g. a target cell of a higher eukaryote, particularly mammals, and more specifically human cells, or injecting them in a model of animal infection, the expression of heat shock (stress) genes and the accumulation of the coded proteins (stress proteins or HSPs) and that of other species-specific gene products, the regulation is altered as a consequence of the coded modification of MPS. Among others, we refer also to genes whose regulation is mediated by signaling transduction pathways. Therefore, as a result of this procedure, pathogens became non-virulent (attenuated, live microorganisms) that can be used for vaccine production.

### FIELD OF INVENTION

The present invention is related to a method to generate non virulent microorganisms from pathogenic ones through permanent genetic modification of their biological membrane. In particular the method of the invention provides a modification of the physical state and/or dynamic state of the membranes in order to obtain modified pathogens that can be used for vaccine production. The invention also refers to the vaccines so obtained.

### TERMS USED IN THE FOLLOWING DESCRIPTION

MPS: Membrane physical state

Cloning Vector: DNA molecules that contain the entire genetic information that allows them to replicate when transfected in a host.

Desaturase Genes: gene coding for desaturase enzymes that introduce a double bond in specific positions (Δ⁶, Δ⁹, Δ¹²etc.) of acyl chains of saturated fatty acids (SFA) to form unsaturated fatty acids (UFA).

Differential Scanning Calorimetry: It is a technique to study the thermotropic behavior of biological membranes. As the temperature of biomembranes is raised through that of gel to liquid crystalline phase transition of its lipid constituents (or domains, with specific lipid head groups and fatty-acyl chains), the mobility of the hydrocarbon chains increases and a corresponding rise in heat absorption occurs. Gel-to-fluid lipid phase transitions are normally completed below or around growth temperature of the organisms. High-temperature thermal transitions are generally attributed to protein denaturation and, in contrast to the lipid transition, are irreversible.

Gene expression: This term indicates an overall process by which an organism synthesize a protein codified by a specific gene through an intermediate molecule, mRNA.

Heat shock genes (stress genes): ubiquitarious genes that are transcriptionally activated rapidly when cells are exposed to a sudden increase in temperature and/or to various forms of stresses. Inducibility by stress is obtained by the presence of specific cis elements in the promoter region of these coding sequences (e.g. heat shock element, HSE).

Heat shock proteins (HSPs or stress proteins): the protein product of heat shock genes rapidly accumulated by a cell after exposure to stress and whose functions include: assign the proper folding of nascent polypeptides, targeting of denatured proteins (misfolded), protection of mitochondrial and chloroplasts functions, mRNA maturation, their insertion in membrane to protect MPS, etc.

Integral (or intrinsic) membrane proteins: Any membrane protein that, partially or totally, interacts with the hydrophobic region of the phospholipid bilayer and that can be extracted from membrane only by detergents.

Macrophages: cells present in the blood, lymph and other tissues. Their functions is to phagocyte and destroy pathogens. Some macrophages are responsible of B and T lymphocytes activation.

Membrane: semi-permeable barrier that surrounds eukaryotic and prokaryotic cells, organelles (e.g. mitochondria, chloroplasts, endoplasmic reticulum, nuclei, etc), that is composed by a lipid bilayer (phospholipids, glycolipids and sterols) in which intrinsic membrane proteins or associated proteins are present. All membrane undergo cell specific changes in their MPS as a result of the genetic manipulation described in the present invention.

Membrane fluidity. A widely used but subjective term that describes the relative diffusional motion of molecules within membranes. Fluidity is used rather than viscosity, because membranes are planar, asymmetric structures, and their properties are not comparable to bulk phases. The term fluidity is meant to convey the impression of lateral diffusion, molecular wobbling and chain flexing, that are found in functional membranes where the lipids are in the fluid-crystalline lamellar phase.

Membrane order. The motional movement of molecules or molecular domains within the membrane. Membrane order can be quantified by estimating the motion of paramagnetic probes and calculating an order parameter from the ESR or NMR spectrum.

Non-lamellar phases. Non-bilayer arrangements of lipids in aqueous media. These can be hexagonal (H_{I}) or inverted hexagonal (H_{II}) arrangements; H_{I} phase is seldom found in membranes.

PCR: (polymerase chain reaction): technique to synthesize in vitro large amounts of specific nucleotide sequences by the use of specific oligonucleotide primers complementary to sequences of the target gene using special termostable DNA polymerases.

pG13: plasmid containing the E. coli green fluorescence protein (GFP) gene under the transcriptional control of a M. marinum promoter (named PG13, acc. # AF092842). PG13 is a σ⁷⁰-like promoter, found both in M. marinum and M. tuberculosis, whose induction in macrophages is about 40 times higher than that seen with mycobaterial hsp60 promoter.

pNir: plasmid inducible anaerobically in vivo containing the pNirB promoter of Escherichia coli that is regulated by the bacterial protein FNR which is activated in anaerobiosis (Dunstan et al, 1999).

Promoter: a specific DNA region in which RNA polymerase initiates transcription. The promoter region contains a recognition site for the enzyme RNA polymerase.

Signaling transduction pathways: Conversion of a signal from a physical (e.g. or temperature, osmolarity) chemical (e.g. hormones) form into an other. In cell biology, this term is referred to the sequential process initiated by the interaction of a chemical factor with a membrane or cell receptor or a physical effect on membrane that culminates in one or more specific cell response (e.g. gene transcriptional activation of sequences under this control).

Transformation: method to obtain proteins through DNA recombinant techniques that requires the cloning of a gene coding for a given protein and where “cloning” means isolation, purification and sequencing of the gene coding for that protein. Once cloned, the nucleotide sequence can be inserted in an appropriate expression vector and the obtained DNA recombinant molecules can be introduced in a microorganism in which the gene is simultaneously replicated with the host DNA. The gene can eventually be re-isolated with standard techniques of molecular biology.

Virulence genes: these genes are defined as those genetic traits that code for molecules toxic for the host (toxins) or for protein products essential for the survival of an intracellular pathogen within the host niche. Thus, several genes coding for these traits are likely to be regulated in response to physical and/or environmental changes such as different forms of stresses.

### BACKGROUND ART

The Heat Shock Response, or stress response, is one of the better studied homeostatic cell responses, mainly involved in the maintenance of cell functionality in response to diverse environmental stresses and/or in pathologic states. This response is mediated by a rapid increase of the transcription of those genes that codify for the stress proteins (Lindquist. 1986). It has been largely demonstrated that such increase in mRNA synthesis of stress genes, and the relative intracellular accumulation of HSPs, is associated with the acquisition of thermotolerance, with protection to subsequent exposure to other forms of stresses or in pathological conditions, etc. (Singer & Lindquist 1998; van Eden & Young 1996). It has been demonstrated that the primary sensor(s) of temperature variations, and in general to other forms of stresses, is (are) localized in the membrane (Carratu et al 1996; Horvath et al 1998, Vigh & Maresca, 1998; Suzuki et al 2000, Piper et al 2000; Torok et al 2001; Vigh & Maresca, 1998). Further, recent studies have shown that an abrupt temperature change or exposure to other forms of stress, determine a physical re-organization of lipid and protein membrane components (Slater et al 1994), that is followed by a specific gene response aimed to compensate variations in MPS. Thus, a cross-talk between changes in MPS and regulation of gene expression exists, particularly for heat shock genes. We have focused our attention on the crucial role of membranes as primary targets of heat stress and have attempted to understand how proper lipid/protein interactions within the membrane determines the transcriptional regulation of HS genes. Such molecular interactions have been shown to be critically involved in the conversion of physical and chemical signals from the environment into sequential processes culminating, in a specific manner, in the transcriptional activation of stress regulated genes. In turn, the interactions between certain HSPs and specific regions (domains) of membranes remodel the status of membrane physical state (overall phase state, order, permeability, etc.). We have shown that the specificity of gene expression is obtained by the uneven distribution of these membrane domains that precisely sense biological and physical environmental regulating signals and different forms of stresses. These studies have strongly modified our vision of the functions of biological membranes. We proposed that the composition, organization and physical state of membranes play central and determining roles in the cellular responses during acute heat stress and pathological states (Vigh & Maresca, 1998).

Among the agents responsible for an appropriate MPS, we mention the desaturase enzymes, that through their enzymatic activities, control membrane phospholipid composition. Desaturases are enzymes that introduce double bonds in SFA to form UFA. The ratio SFA/UFA is one of the critical factors that determine an appropriate MPS in all cells (Cossins, 1994). Recently, it has been shown that synthesis of inducible HSPs is controlled by abrupt and local variations of a number of factors that include:
Membrane lipid composition
Membrane lipid/protein interactions
Membrane lipid dynamic (MPS modification) (Vigh et al 1998, Vigh & Maresca, unpublished, 1998)

Thus, change of the MPS under stress condition re-determines the threshold value at which HSPs are normally synthesized.

Intracellular pathogens, such as Salmonella typhimurium, Mycobacterium tuberculosis, Mycobacterium marinum Histoplasma capsulatum, trypanosomes, etc., at the onset and during infection of macrophages and of other cells, induce a genetic response through transcriptional activation of stress genes and other sets of species-specific genes, here defined, in general terms, as virulence genes. The gene products are directly involved in the mechanisms of invasion/adaptation, operate in a coordinate fashion, and are responsible of the capacity for the pathogen to invade, replicate and induce disease (virulence genes) in the host (Groisman & Ochman 1997). This vast, coordinated and generalized genetic response allows intracellular pathogens such as S. typhimurium, M. marinum to induce the disease avoiding the immune response of the host.

All bacteria, fungi and parasites in general, agents responsible for deadly diseases not yet eradicated, such as salmonellosis, typhoid fever, tuberculosis, histoplasmosis, candidosis, malaria, trypanosomiasis, etc, induce HSPs as an essential part of their response to the conditions encountered in the host at the onset of infection (Groisman & Saier 1990). Hsp70s of eukaryotic pathogens and bacterial Hsp60 (GroEL) are primary antigens, that constitute up to 15% of the cell's dry weight (Feige and van Eden, 1996). Thus, stress genes and those involved in virulence are strictly interconnected and genetically coordinated (Groisman & Ochman 1997). While the details of the regulation of these virulence genes have not elucidated yet, it is known that the proper expression of these sequences is also under the control of an appropriate amount of HSPs that are synthesized at the onset of infection. The traditional methods to fight pathogens are believed to be scarcely effective since they are based on the use of vaccines that stimulate an immune response against one or few antigens. For example, some strains of Mycobacterium tuberculosis, the etiologic agent of tuberculosis, have become progressively resistant to the available antibiotics. Since the identification of new effective non toxic antibiotics requires many years of experimental work and huge investments, it is urgent to produce new types of vaccines against the new resistant strains.

Regarding Salmonella, the present vaccines are:
Vaccines with attenuated strains injectable parenterally;
Vaccines made of purified bacterial capsules that can be injected parenterally;
A Ty21 vaccine composed of attenuated strains of Salmonella administered orally.

Such vaccines, though give a permanent immunity, have several disadvantages. For example, it is not determined that vaccines made of killed bacteria are virtually free of live cells, and that the attenuated ones do not revert to the virulent form. It is also possible that vaccines obtained with killed organisms contain substances, cells or traces of medium that may be toxic for humans. Further, vaccines with killed organisms often have high incidence of side effects, that combine with a low level of protection. Vaccines made of infective attenuated particles, obtained by a rapid thermal or chemical treatment, may contain antigens whose physiological protein folding is modified (denatured) thus altering the natural immunological response.

Vaccines not involving the use of attenuated strains are generally composed of a single or a few antigens that give only a partial and incomplete protection compared to the natural immunological response.

In general, this has a drawback that results in the low efficacy of today's vaccines, since, in man, as in mammals in general, and more in general in higher eukaryotes, the natural mechanisms of protections against infective agents comprise a complex immunological response against the entire microorganism and all its combined antigens.

Authors have now hypothesized, and experimentally proved, that, altering the physiological MPS and the stress response of pathogens, it is possible to attenuate strains to be utilized for vaccine production, voided of the above mentioned side affects.

### SUMMARY OF THE INVENTION

It is an object of the present invention to provide a method for the permanent genetic modification of microorganism's MPS, in particular intracellular pathogens (e.g. bacteria, fungi, parasites) such as S. typhimurium, M. marinum, H. capsulatum, trypanosomes, etc. with the aim of altering the synthesis and accumulation of HSPs in such pathogens at the onset of infection and of those species-specific gene products, whose regulation is modified as consequence of MPS, including genes whose activation is mediated by signaling transduction pathways (e.g. c-fos, fos B, junB, junD, MAP kinase, genes), where the host can be a target cell such as a macrophage, cells of higher organisms in general, or of mammals or of humans. Therefore, as a result of this modification, pathogens become non-virulent (attenuated).

Another object is the use of these attenuated strains to produce vaccines.

A further object is the development of a new class of vaccines for human use, and for animals in general, comprising the modified pathogens.

A further object is the method for producing attenuated non-virulent strains of pathogenic microorganisms, such as S. typhimurium, M. marinum, H. capsulatum, etc. through the transformation with a vector carrying and express a gene coding for Δ¹²-desaturase of Synechocystis PCC6803 or for other desaturase genes of Salmonella or for other prokaryotic and eukaryotic microorganisms (e.g. Δ⁹-desaturase of S. cerevisiae or of H. capsulatum) or for genes coding for integral membrane proteins that cause a perturbation of MPS (lipid phase transitions, permeability). With the genetic modification produced according to the invention it is possible to obtain, for example in Salmonella, an increases in the ratio protein/lipid of total membrane (mixture of outer and inner or cytoplasmic membranes) from about 100 (virulent strain) to about 170 (strain genetically modified) (FIG. 4) or, for example in M. marinum a decrease of at least 4° C. in the mutant strain of the major lipid phase transition of its outer cell membrane. The method can be extended. to other intracellular pathogens listed herein below as a non-limitative example.

Further objects will be evident form the following detailed description of the invention.

### SHORT DESCRIPTION OF THE FIGURES

FIG. 1 Vector (panel A) containing Δ¹²-desaturase gene (SEQ ID N. 1) of Synechocystis PCC6803 (accession number X53508, CyanoBase http//www.Kazusa.or.jp/cyano/) (panel B) into the restriction site Ndel of pNir plasmid under the control of PNir promoter. Ndel restriction site was generated by PCR using appropriate primers (desA-Ndel 3′—SEQ ID N. 3 and desA-Ndel 5′—SEQ ID N. 4) (panel C). FNR is the DNA binding site for FNR protein which induced by bacteria in anaerobiosis and activates downs stream transcription from the PNir promoter.

FIG. 2 Expression of Δ¹²-desaturase gene of Synechocystis PCC6803 in S. typhimurium grown at 37° C. and 15 min after heat shock from 37° C. to 41°, 45° ad 47° C. as shown by Northem blot. Endogenous Δ¹²-desaturase mRNA is not measurable in cells transformed with the empty vector only (pNir).

FIG. 3 Denaturing gel of protein extracts (7 and 15 μg of proteins) purified from a fraction of a sediment of a preparation of a lysed Salmonella, expressing in anaerobiosis Δ¹²-desaturase gene or containing the empty vector (a=S. Typhimurium (pNir) grown to late log phase in O₂, b=S. Typhimurium (pΔ¹²) grown to late log phase in O₂, c=S. Typhimurium (pNir) grown 20 hrs without O₂, d=S. Typhimurium (pΔ¹²) grown 20 hrs without O₂). The arrow shows Δ¹²-desaturase protein. Δ¹²-desaturase protein is not identifiable in the soluble fraction of the bacterial lysis. Cells are grown in oxygen to late log phase to deplete oxygen (anaerobiosis) or in anaerobiosis

FIG. 4 Change in the Protein/Lipid ratio in total membrane of Salmonella containing pNir::Δ¹² construct -des or pNir only.

FIG. 5 Coomassie gel: SDS PAGE (17%) of Salmonella outer membrane proteins. Cells were grown at 30° C. Western blot: Identification of Δ¹²-desaturase in the outer membrane fraction of Salmonella containing the pNir::Δ¹² construct or pNir only.

FIG. 6 Antibody of Δ¹²-desaturase of Synechocystis was used in 1:2000 dilution. As a positive control Synechocystis total cell extract was applied (Syn.).

FIG. 7 Change in the membrane permeability in Salmonella containing pNir::Δ¹² construct -des or pNir only. Overproduction of the membrane protein Δ¹² desaturase induces an elevated outer membrane permeability in Δ¹²-transformed Salmonella strain between 25°-40° C. (cells grown at 30° C.).

FIG. 8 Change in the membrane permeability in Salmonella containing pNir::Δ¹² construct -des or pNir only. Set of data are the same of FIG. 7 and are presented as a ratio between the two sets of values.

FIG. 9 DSC Effect of the overproduction of Synechocystis Δ¹²-desaturase on the thermotropic behavior of the outer membranes examined by differential scanning calorimetry. Raw data of first and second heating scans. Normalized data of first heating scans. Membranes were isolated from cells grown at 30° C.

FIG. 10 DSC Major signals of peaks of FIG. 9 are amplified and shown.

FIG. 11 Expression of DnaK (hsp70) gene in Salmonella containing the pNir:Δ¹² construct-des or pNir only, by Northern blot. Cells were grown at 30° C. and heat shocked at 37°, 39° and 43° C. for 15 min.

FIG. 12 Expression of DnaK (hsp70) gene in Salmonella containing the pNir:Δ¹² construct-des or pNir only by Northern blot. Cells were grown at 37° C. and heat shocked at 41°, 43°, 45° and 47° C. for 15 min.

FIG. 13 Expression of GroEL gene (hsp60) of Salmonella containing pNir:Δ¹² construct with -des or pNir only, by Northern blot. Cells were grown at 30° C. and heat shocked at 37°, 39° and 43° C. for 15 min.

FIG. 14 Expression of GroEL gene (hsp60) gene of Salmonella containing pNir:Δ¹² construct with -des or pNir only, by Northern blot. Cells were grown at 37° C. and heat shocked at 41°, 43°, 45° and 47° C. for 15 min.

FIG. 15 Quantification of Salmonella DnaK induction containing pNir:Δ¹² construct -des or pNir only. Data are those of FIGS. 11 and 12.

FIG. 16 Northern blot showing the expression of DnaK (hsp70) and lbpB (hsp17) genes in Salmonella containing the (Δ¹²) pNir::Δ¹² construct or Nir (pNir; Control) by Northern blot. Cells were grown at 30° C. and heat shocked at 45° C. for 15 min.

FIGS. 17 (a-c) Curve of toxicity of benzyl alcohol. Effect of 30 min exposure to increasing concentration (5 to 80 mM) benzyl alcohol (BA) on the growth of S. typhimurium.

FIG. 18 Expression of Salmonella DnaK induction containing pNir:Δ¹² construct-des or pNir only or treatment with 50 mM BA (Northern blot).

FIGS. 19 (a-f) Effect of transformation of S. typhimurium with pNir:Δ¹² construct-des or pNir only (virulent strain) on macrophage infection. Macrophages were infected with a macrophage/Salmonella ratio of 1:1 (MΦ/S.th. 1:1) up to 30 min. After macrophage lysis, Salmonella was recovered and aliquots were plated on agar to determine survival. Cells genetically modified are no longer virulent.

FIGS. 20(a-e) Treatment of S. typhimurium with 50 mM BA and its effect on macrophage infection. Macrophages were infected with Salmonella (MΦ/S.th. 1:1) up to 60 min. After macrophage lysis, Salmonella was recovered and aliquots were plated on agar to determine survival. After an initial shock cells recover by restructuring the cells.

FIG. 21 Vector pG13Δ¹² containing Δ¹²-desaturase gene (DesA) of Synechocystis PCC6803 under the control of PG13 promoter. The gene was cloned in the Ndel restriction site of the vector.

FIG. 22 Expression of Δ¹²-desaturase gene in M. marinum transformed with pG13Δ¹² containing Δ¹²-desaturase gene of Synechocystis. No expression Is detectable in control cells.

FIG. 23 Growth of M. marinum transforned with pG13 and pG13Δ¹² containing Δ¹²-desaturase gene of Synechocystis PCC6803 under the control of PG13 promoter.

FIG. 24 Change in the Protein/Lipid ratio in total membranes of M. marinum and transformed with pG13Δ¹² containing Δ¹² -desaturase gene of Synechocystis PCC6803 under the control of PG13 promoter.

FIG. 25 Survival of M. marinum transformed with pG13Δ¹² containing the Δ¹²-desaturase gene of Synechocystis PCC6803 under the control of PG13 promoter is abolished while growth of control cells recovers as expected.

FIG. 26 Changes in transcription of hsp70 gene of H. capsulatum virulent strain genetically modified (D3) (Northern blot).

FIG. 27 Comparison of the capacity of virulent G217B strain and modified D3 of H. capsulatum to grow intracellularly in murine macrophages. D3 strain does not multiply within macrophages.

FIG. 28 Survival curves of mice inoculated with virulent G217B strain and with non-virulent D3 and then challenged with normal virulent G217B strain.

### DETAILED DESCRIPTION OF THE INVENTION

The method according to the present invention can be performed using genetic engineering techniques, as described, e.g., by Sambrook et al. (1989) by constructing vectors containing promoters capable to drive the expression of the gene(s) of interest during infection (e.g. in anaerobiosis, or in cells such as macrophages or other mammalian cells, in the human or in the animal host) in the pathogens that will be used eventually to obtain specific vaccines. The modified strain can then be utilized to produce injectable vaccines, or exploitable orally or via nasal spray.

The methods of the invention can be summarized according to the following main steps:
construction of an appropriate vector for each specific pathogenic organism to regulate the expression (constitutive or inducible) of a gene, under the control of a suitable promoter, and the relative production of the protein product, the said protein being able to modify the membrane of a pathogen, e.g. an enzyme that modifies the level of saturation within fatty-acyl chains of membrane lipids or that of a membrane integral protein able to translocate to and interact with pre-existing lipoprotein complexes of the cell membrane; the vector may contain, for example, Δ⁹-desaturase or Δ¹²-desaturase gene or other desaturase genes or genes coding for integral membrane proteins or their derivatives;
genetic transformation of a virulent strain (the pathogen) with the mentioned specific vectors for each pathogenic organisms;
expression of the protein product capable of interacting directly (integral protein) or indirectly (desaturase) with the membrane of the pathogen;

An in vitro test to verify the effect on transcription of stress genes, desaturase, integral membrane protein, and/or of virulence genes and/or of signaling pathways (e.g. by Northern blot) can be performed on the virulent pathogen.

The non-virulent strains so obtained, can be processed according. to known methods in order to be utilized as active principle in effective amounts to produce a vaccine.

The preparation of a vaccine using modified pathogenic strains according to this invention can be performed by the skilled man. It is possible to set up a procedure to produce vaccines containing eccipients, adjuvants and other conventional agents that can be used for administration, for example intradermal, intramuscular, intravenous, mucosal, vaginal, oral, rectal, nasal use.

This genetic procedure is applicable in particular to all intracellular pathogens, prokaryotes and eukaryotes, but does not exclude extracellular pathogens. Examples of pathogens that can be used to apply the method of the invention are those mentioned in the present not exhaustive list:

Strict intracellular bacteria: Chlamydia species (pneumoniae and trachomatis, Coxiella burnetii, Ehrlichia chaffeensis, Rickettsiae.
Facultative intracellular bacteria: Legionella pneumophila, Mycobacteria (M. tuberculosis, M. leprae), Nocardia species (mycetoma), Bartonella species, Brucella species, Francisella tularensis, Listeria monocytogenes, Salmonella species, Shigella species.
Other bacteria: Borrelia burgdorferi (Lyme disease), Treponema pallidum, Campylobacter, Haemophilus influenzae Klebsiella pneumoniae, Leptospira interrogans, Neisseriae species, Staphylococci, Streptococcus pyrogenes, S. agalactiae, S. pneumoniae, Yersiniae, Bacillus anthracis.
Fungi: Aspergillus fumigatus, Candida species, Cryptococcus neoformans, Histoplasma capsulatum, Pneumocystis carinii.
Parasites: Entamoeba histolytica, Leishmania species, Plasmodium falciparum and vivax and other species, Toxoplasma gondii, Trypanosoma cruzi.

Combinations and/or mixtures of different kinds of pathogens can be possible within the scope of the present invention.

The method according to this invention allows to obtain an altered MPS and a decrease in the amount of synthesized HSPs in the pathogen when they associate with the host, a host that can be a macrophage, or, in general, another type of a cell of a higher eukaryote, particularly a mammal, more specifically a human being. The decrease in the amount of HSPs is controlled by perturbation of MPS, that is induced with the method described in this invention and that, as an example, may imply a change in:
membrane lipid composition,
protein-to-lipid ratio within membranes
membrane permeability and thermal phase transitions

Therefore, the genetic modification of MPS according to the invention, under the stress condition encountered by the pathogen (when the pathogen interacts and is internalized by the host's cells that it infects), establishes a new (different) stress threshold at which stress (heat shock or hs) genes are normally transcribed. Thus, modification of MPS “freezes” the pathogen in a physiological and immunological competent state, with the entire set of not modified antigens. The modified pathogen is not capable of synthesizing, during infection, in the appropriate time, the proper amount of specific proteins that are involved in the process of adaptation of the pathogen to the conditions present in the host and that allow its invasion, multiplication and eventually to cause disease. The modified pathogen has a reduced capacity to adapt to the host's conditions and, therefore, the disease does not occur.

With this method, the antigens of the attenuated pathogens are not modified structurally and thus the pathogens are fully immunocompetent. Further, these modified intracellular pathogens are not able to induce properly the genetic and definite species-specific program (e.g. new specific antigens and proteins) necessary to avoid the host immune response. Therefore, the method of the invention allows the production of strains of pathogens attenuated in their mechanism of virulence but fully immunocompetent. The live attenuated vaccines so obtained, represent the best protection from intracellular pathogens.

According to a particular embodiment of the invention, we describe in the examples the main steps relative to the production of a non virulent strain of S. typhimurium, M. marinum and H. capsulatum and their use for vaccine production. The production of attenuated non virulent strains such as S. typhimurium, M. marinum and H. capsulatum is obtainable through transformation of the corresponding pathogenic microorganisms with a vector that carries and expresses the Δ¹²-desaturase gene of Synechocystis PCC6803 or other desaturase genes of Salmonella or of other prokaryotic and eukaryotic organisms (e.g. H. capsulatum or S. cerevisiae Δ⁹-desaturase, etc.), or other genes coding for integral membrane proteins that are able to cause perturbation of MPS of the pathogens. In particular, integral membrane proteins intercalating in cellular membranes (outer and inner or cytoplasmic, nuclear, mitochondrial, etc), alter the pre-existing protein/lipid ratio and thereby modify their permeability and thermal phase transition profile closely linked to the ability of a membrane to function properly in a given temperature range). Such modification causes a diffuse or localized modification of MPS as a consequence of the expression of exogenous genes coding for integral membrane proteins with an effect similar to that determined by the enzymatic activity of desaturases that modify these parameters by alteration of the SFA/UFA ratio.

The method comprises the following steps:
construction of an appropriate vector for a given pathogenic agent containing a gene whose product modifies directly or indirectly the MPS of a pathogen;
genetic transformation of virulent strains of the pathogenic bacterium S. typhimurium with cyanobacterium Synechocystis PCC6803 Δ¹²-desaturase gene and of virulent H. capsulatum G217B strain (G217B: ATCC 26036, American Type Culture Collection, Rockwille, Md.) with the plasmid containing the Δ⁹-desaturase gene under the control of a promoter driving the expression of the downstream gene during the infective stage of the pathogen(s), e.g. up-regulated promoter of the Downs strain.
Over-expression of Δ¹²- or of Δ⁹-desaturase gene or other desaturase genes or genes coding for integral membrane proteins;
Enzymatic desaturase activity;
Translocation of the protein product into membranes;
Alteration of MPS via enzymatic activity and/or insertion of the protein in membrane.

The modifications obtained with the described method induce an alteration of the pattern of expression of stress genes and that of other genes such as those responsible of virulence and/or of genes involved in the adaptation to the conditions present in the host and that are implicated in cell survival and virulence of the pathogen and/or genes regulated by signal transduction pathways under the control of MPS. Perturbation of MPS and the changes in gene expression are associated with the loss of virulence of S. typhimurium and H. capsulatum during macrophage infection and in animal model of infection. The attenuated strains so obtained induce immune protection against virulent strains of S. typhimurium and H. capsulatum.

The results obtained with the method of the invention show that a perturbation of membrane genetically obtained over-expressing a desaturase gene or membrane proteins that locally or diffusely alter MPS or treating membrane with molecules that perturb MPS, cause a significant modification of the capacity of Salmonella, M. marinum (and other prokaryotic and eukaryotic intracellular pathogens) to accumulate an appropriate amount of stress proteins.

Membrane perturbations so obtained cause the permanent loss of virulence of Salmonella, M. marinum and H. capsulatum (and that of other pathogens) when strains genetically transformed are used to infect a macrophage cell lines (J774) or murine macrophages or other cells or to infect a susceptible animal to the infection or in humans.

The genetic procedure described in the examples allows the production of non-virulent strains, such as S. typhimurium and H. capsulatum thus permitting the development of vaccines against these pathogens. This technology can be applied to other intracellular pathogens either prokaryotes or eukaryotes (see the above mentioned list) to obtain other attenuated strains to develop other vaccines.

The following examples and figures are presented to better show the invention and they should not be considered as limitative of the scope thereof.

### EXAMPLE 1

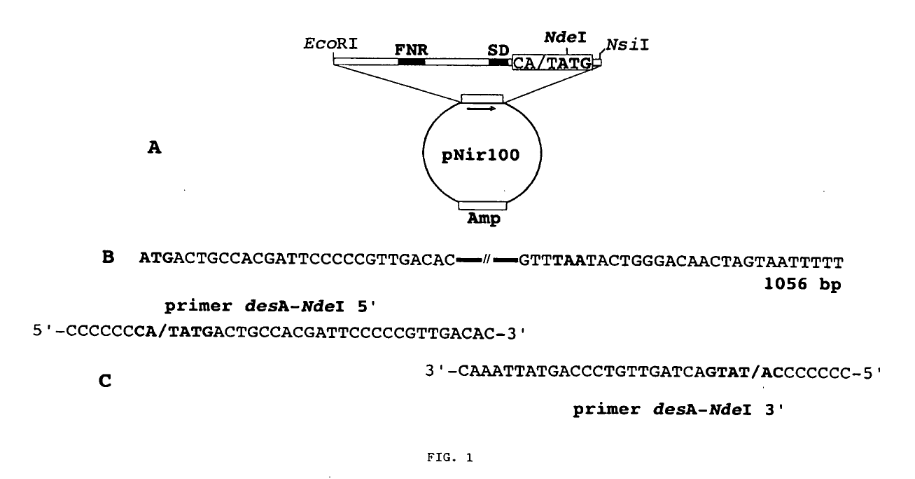

The entire Δ¹²-desaturase gene (SEQ ID N. 1) of cyanobacterium Synechocystis PCC6803 has been cloned by PCR and inserted in pNir vector (FIG. 1) under the control of the inducible E. coli PNir™ promoter in anaerobiosis (Dunstan et al 1999) and it was used to transform wild type virulent S. typhimurium strains. Other promoters that can drive the expression of a downstream gene can substitute for PNir, both in normal growth or during infection of macrophages and in an animal susceptible to the infection.

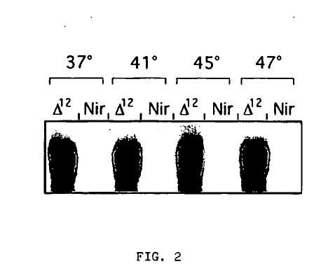

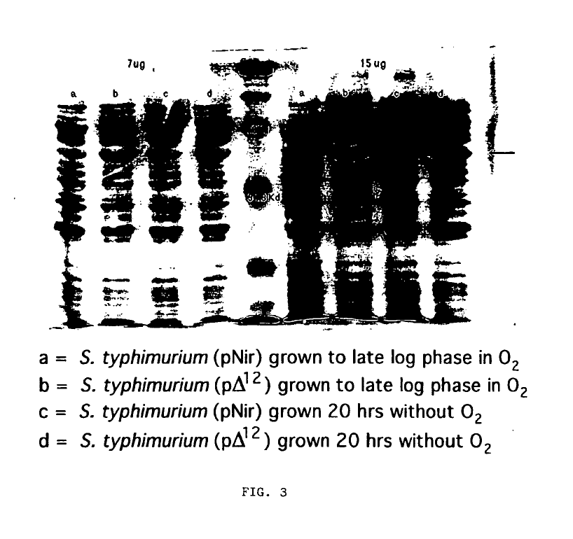

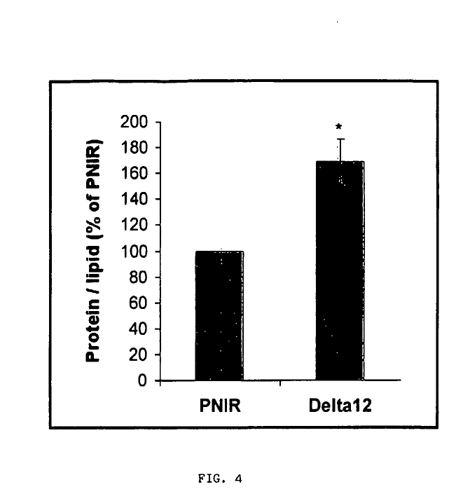

Transformed strains are organisms genetically and permanently modified with pNir and that over express, in anaerobiosis, elevated levels of Δ¹²-desaturase mRNA (FIG. 2) that is translated in high level of the coded protein (FIG. 3). Δ¹²-desaturase gene codes for high level of the protein (SEQ ID N. 2) at least up to 47° C. (FIG. 2). is Inner, outer and total membranes were isolated from Salmonella as described (Janoff et al., 1979). Lipid content of isolated Salmonella total membranes were determined by GC analysis using heptadecaonic acid as internal standard. Protein concentration was measured by the method of Lowry modified for membrane proteins (Peterson, 1983). FIG. 4 shows the determination of the total membrane Protein/Lipid ratio in control and transformed Salmonella cells. The exogenous expression of the Δ¹²-desaturase gene and the localization of its protein product in the total membrane causes a strong increase in the membrane P/L ratio, with at least 60% increase that determines membrane destabilization and alteration of the signal that induces heat shock gene transcription and possibly of other genes.

Protein Analysis of Isolated Salmonella Outer Membranes.

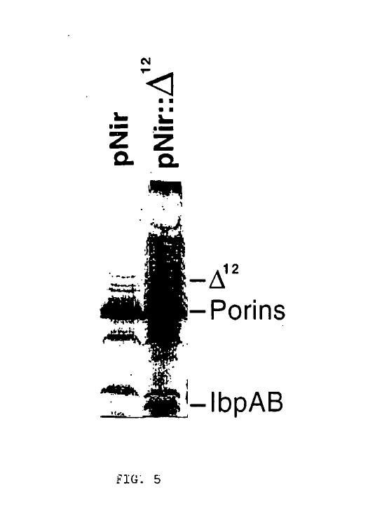

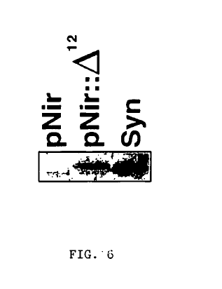

SDS PAGE analysis of outer membrane proteins reveals a significant increase in the amount of proteins in the pNir::Δ¹² strain compared to pNir (Coomassie gel, FIG. 5). Western blot analysis using Δ¹²-antibody of Synechocystis verifies the presence of the desaturase in the outer membrane fraction of pNir::Δ¹² which cannot be detected in the pNir strain (Western blot, FIG. 6). As a positive control Synechocystis total cell extract was applied (Syn.). A large increase in the level of an additional protein band was detected as a result of the over expression of the Δ¹² desaturase (Coomasie gel). This protein band was further characterized by mass spectrometry, and found to consist of two heat shock proteins of Salmonella, lbpA and lbpB (the members of small HSP family).

Identification of sHSPs on the Outer Membrane of pNir::Δ¹² Strains by Mass Spectrometry.

To characterize further the proteins identified in the gels, coomassie blue-stained 1D gel band from Salmonella (pNir::Δ¹²) outer membrane preparation was cut out (MW: <20 kDa). The gel band was subjected to the in-gel digestion protocol (using 0.1 μg trypsin for 7 h at 37° C. following reduction (with DTT) and alkylation (with iodoacetic amide) of the Cys sulfhydril groups. After extraction of the tryptic peptide digest from the gel, purification over C18 ZipTip was performed and the resulting unseparated mixture was analyzed by MALDI-TOF in dihydroxybenzoic acid matrix. Based on MALDI analysis, MS-Fit database search identified two proteins from the mixture:
is heat shock protein lbpB (of Salmonella enterica, NCBI# (03.26.2002): 16762514, MW: 16 kDa). This hit identified 30% of the found m/z values covering 40% of the identified protein. This identification was further confirmed by the PSD spectrum of MH⁺=961.65, identified by MS-Tag database search as ITLALAGFR, [47-55] of the above protein (FIG. 5).
heat shock protein lbpA (of Salmonella enterica NCBI# (03.26.2002): 16762513, MW: 16 kDa) This hit identified another 35% of the found m/z values covering 40% of the identified protein. This identification was further confirmed by the PSD spectrum of MH⁺=1124.58, identified by MS-Tag database search as NFDLSPLYR, [3-11] of the above protein (FIG. 5).
   Perturbation of Membrane Functionality Measured in vivo as a Function of Membrane Permeability.

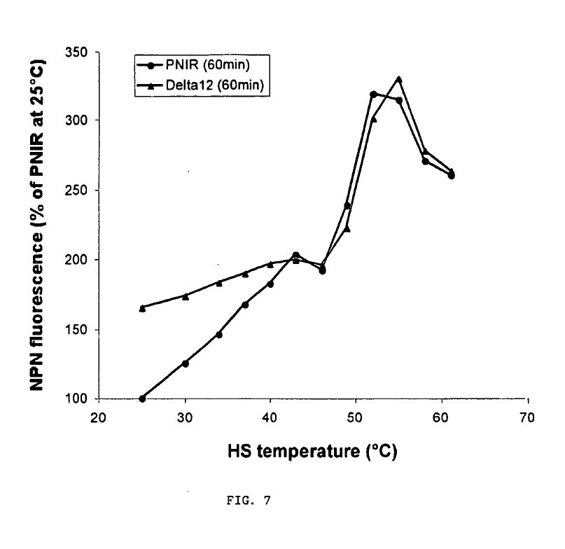

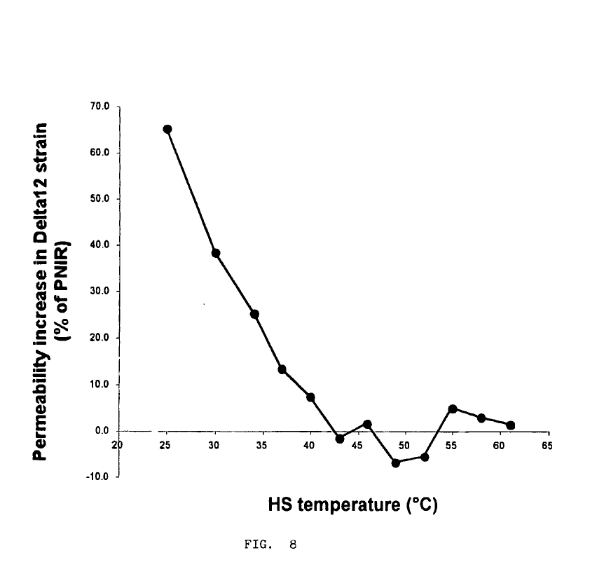

FIG. 7 shows changes in fluorescence of phenylnaphtylamine (NPN). NPN is an uncharged lipophilic dye that fluoresces weakly in aqueous environments, but it greatly increases in nonpolar hydrophobic environments such as in cell membrane. As a result of the outer membrane permeability damage, NPN labels more and more membranes increasing its fluorescence (Tsuchido et al. 1989). In a broad temperature range (between 25° and 55° C.), the over production of Δ¹²-desaturase protein induces membrane destabilization that results in a permanent leakiness of the outer membrane of transformed cells compared to cells containing only the plasmid. The effect of increased leakiness is very significant between 25°-40° C., while it is not present at higher heat shock temperatures. Clearly, above 42-45° C., the heat induced collapse of the membrane becomes predominant over the membrane desintegration caused by protein/phospholipid imbalance detected at lower temperatures. FIG. 8 shows, using the same data of FIG. 7, the effect of over production of membrane-bound Δ¹²-desaturase by inducing an elevated outer membrane permeability in Δ¹²-desaturase-transformed Salmonella strain at 25°-40° C. (cells were grown at 30° C.).

Differential Scanning Calorimetry.

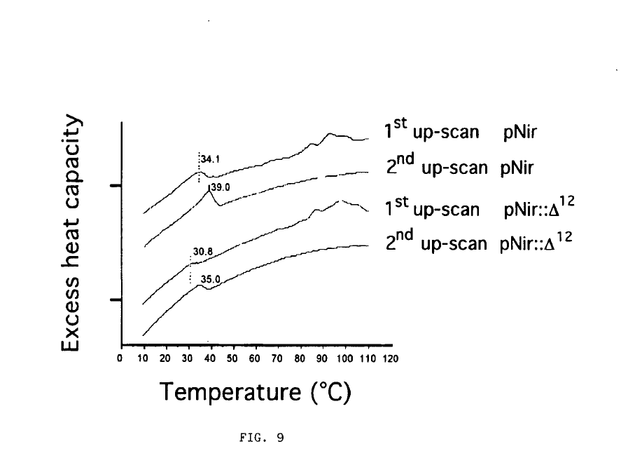

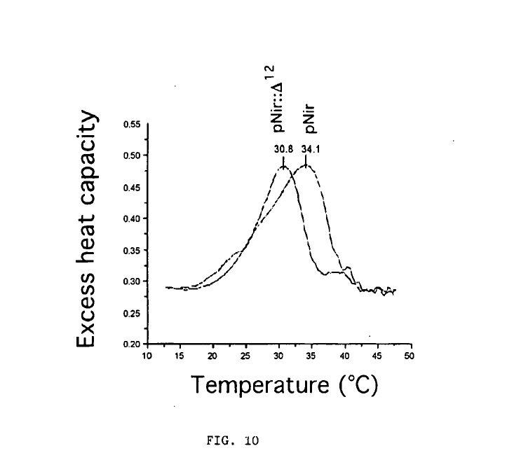

The effect of the overproduction of Synechocystis Δ¹²-desaturase on the thermotropic behavior of the Salmonella outer membranes were examined by differential scanning calorimetry (DSC). In the temperature range between 10° and 65° C., one major endothermic peak was observed in the first up-scan, that appeared repeatedly in the second up-scan as well, whereas several major endothermic peaks appeared in the high temperature range (65°-110° C.). The endothermic peaks in the high temperature range were absent from the second up-scan, a result that suggests that these peaks were originated from irreversible protein denaturation. The reversible endothermic peaks in the 15°-45° C. temperature region correspond to phase transition of membrane lipids (FIG. 9). The midpoint of the transition were at 34.1° C. and 30.8° C. for the outer membranes isolated from pNir and the pNir::Δ¹² strains respectively (FIG. 10). These results indicate that the overproduction of the Δ¹²-desaturase lowers markedly the transition temperature of certain lipid domains in the outer membranes of the cells.

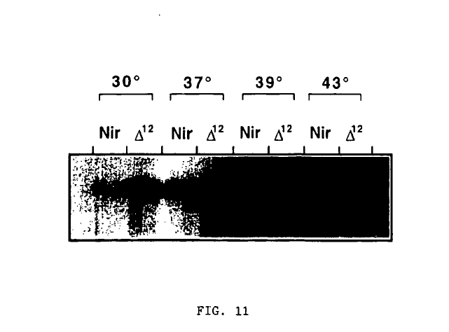

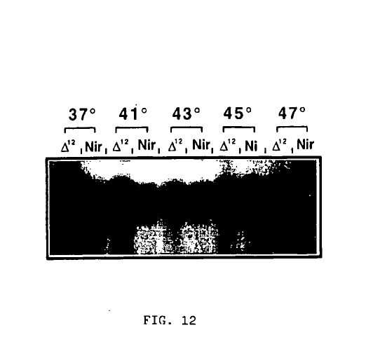

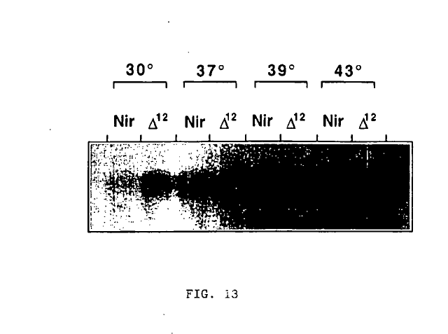

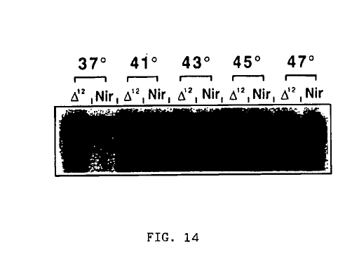

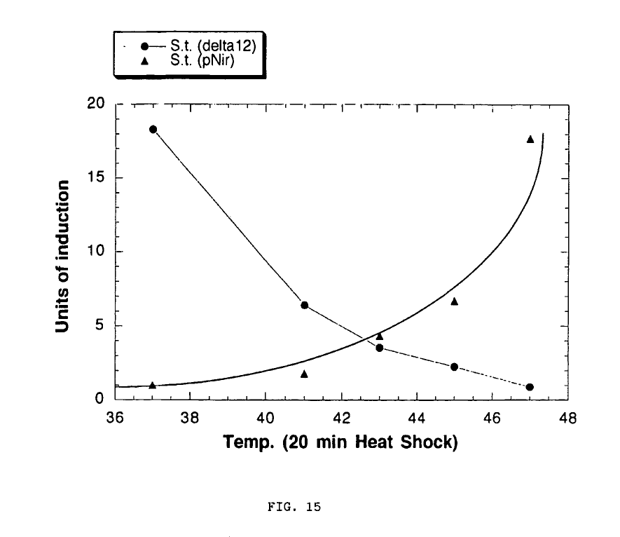

Perturbation of MPS due to the over expression of Δ¹²-desaturase protein re-sets the optimal temperature of expression of heat shock genes (stress genes DnaK (FIGS. 11, 12) and GroEL (FIGS. 13, 14) with a significant change in the pattern of accumulation of HSPs between 30° C. and 47° C. and in the hostile conditions present in the host cells (macrophages and mammals). FIG. 15 shows quantification of DnaK mRNA accumulation under heat shock conditions in cells grown at 30° C. or 37° C. and stressed at different temperatures. Cells over expressing the desaturase gene show a markedly different mode of accumulation of mRNA (pattern opposite to that of wild type cells). Data are obtained from gel analysis of FIG. 11.

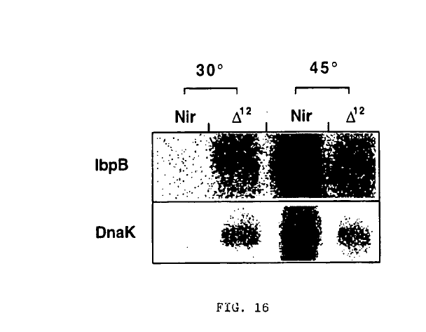

FIG. 16 is a Northern blot showing the similar change in the pattern of accumulation of both DnaK (hsp70) and lbpB (hsp17) mRNAs of genetically modified cells of Salmonella do not induce either genes at the stress temperature of 45° C., while they are expressed at 30° C. Wild type cells have opposite behavior. Overproduction of Synechocystis Δ¹²-desaturase, inactive enzymatically within the cells for the lack of the appropriate substrate under our experimental conditions, inserts itself in the membrane lipid bilayer and cause resetting of the HSR in Salmonella. A substantially higher membrane protein content, ie. an unbalanced protein/phospholipid ratio is found in the membranes of transformed cells. As an additional evidence, the desaturase transformed cells are unable to accommodate properly the extra membrane protein, displaying an altered thermal phase is transition profile and greatly elevated permeability in. their outer membrane even under non-stressed condition. Unbalanced membrane organization (primary event) triggers a complex compensatory mechanism, including alteration of the phase transition temperature of certain lipid domains and the outer membrane association of the small HSP family members, lbpA and lbpB. As a result, the pattern of expression of HSPs is strongly reduced and this in turns affects the proper mechanism of adaptation of the modified strain when it encounters a susceptible host.

### EXAMPLE 2

Membrane perturbation can be obtained also with chemical treatment with drugs such as benzyl alcohol (BA). It produces an effect similar or higher of the level of expression of heat shock (DnaK and GroEL) genes, though its effect is temporary and the example that follows will explain that.

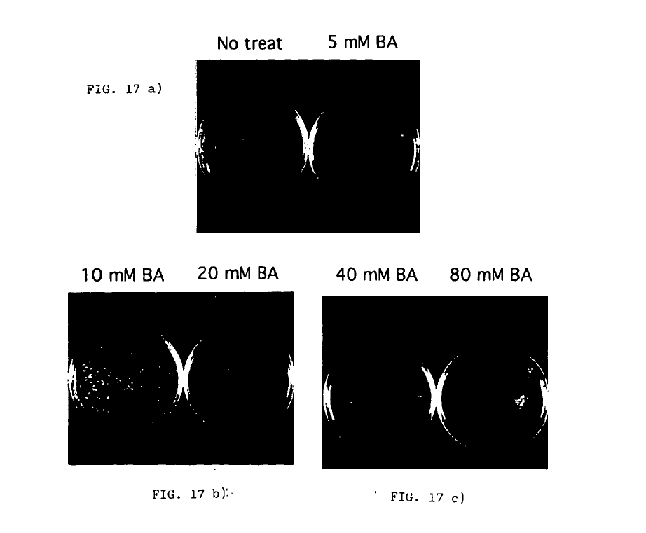

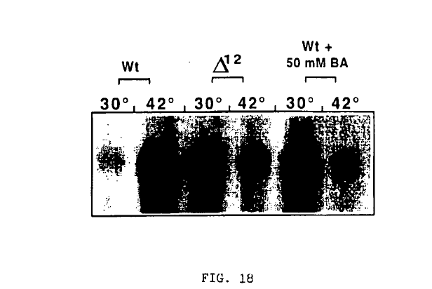

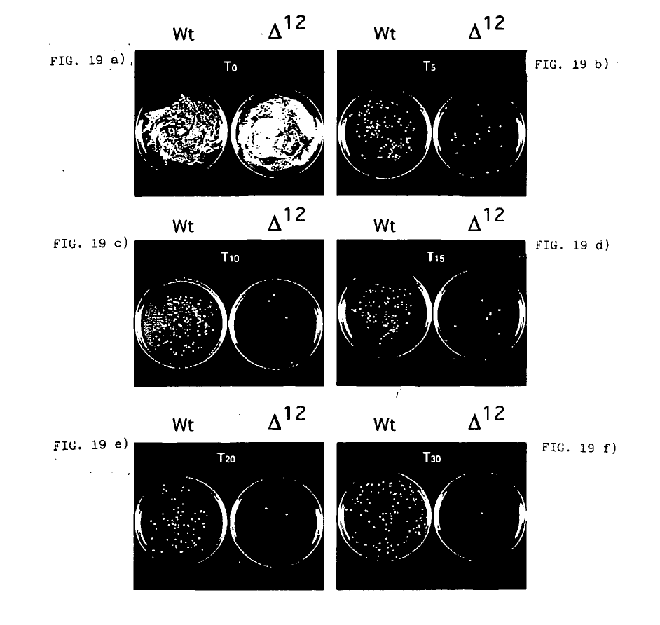

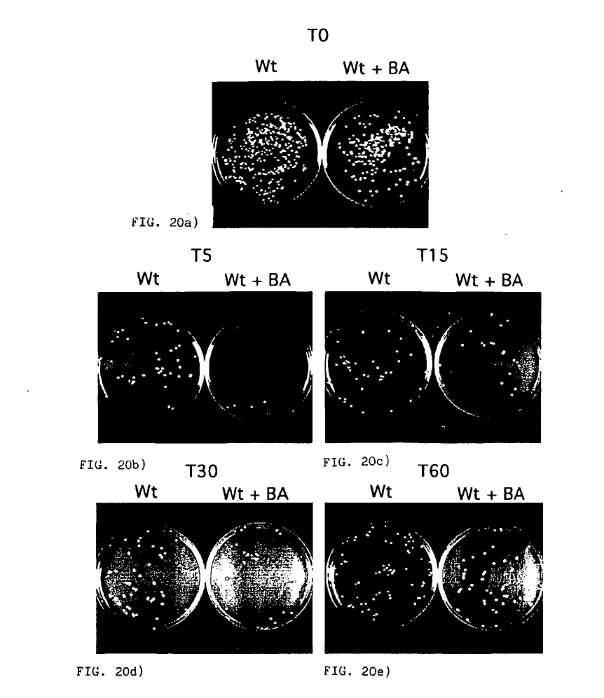

The concentration used (up to 50 mM) have no toxic effect on the growth of Salmonella (FIG. 17). FIG. 18 shows that at the concentration of 50 mM BA there is a significant decrease in the level of accumulation of DnaK at 42° C. when cells are grown at 30° C. These results show that a perturbation of membrane obtained genetically over expressing a desaturase gene or by chemical treatment with BA determine a significant change in the capacity of Salmonella to accumulate an appropriate amount of stress proteins. However:
Membrane perturbation produced genetically (see example 1) causes the full and permanent loss of virulence of Salmonella when it is used to infect a macrophage cell line (J774, FIG. 19);
Treatment of Salmonella with BA reduces its capacity to infect macrophages only temporarily for the removal of BA from the growth medium and for the physiological reconstruction of membrane (FIG. 20).

### EXAMPLE 2.1

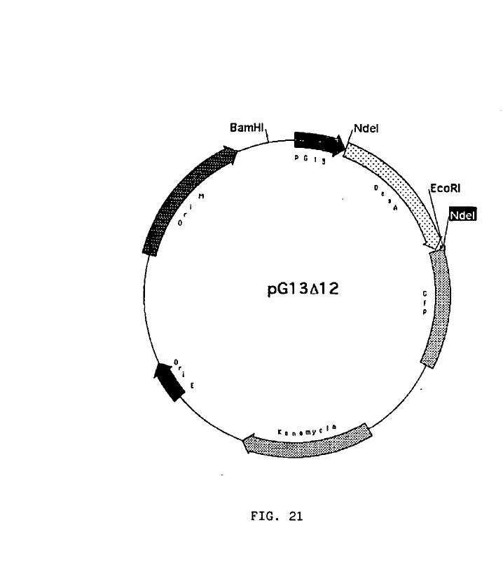

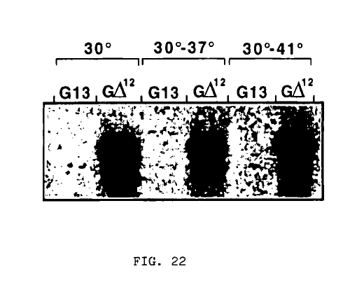

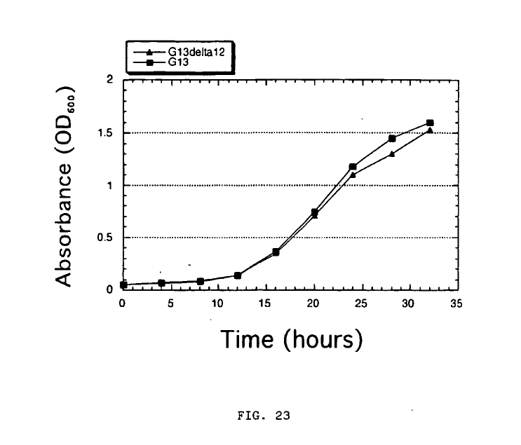

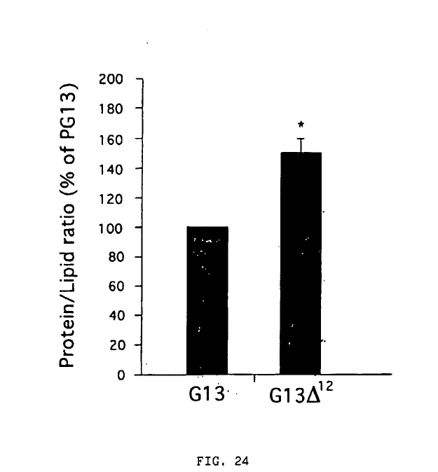

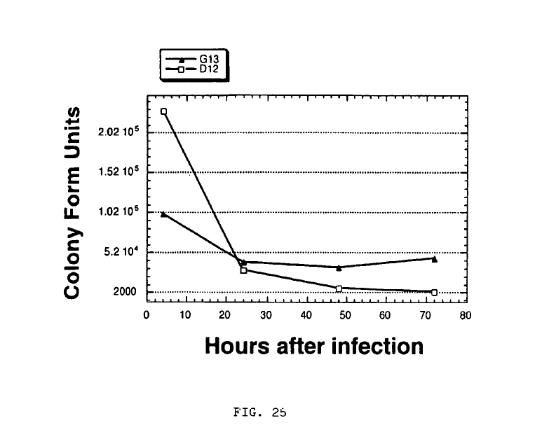

The entire Δ¹²-desaturase gene of cyanobacterium Synechocystis PCC6803 has been cloned into pG13 vector under the control of PG13 promoter (FIG. 21) (Barker et al 1998, 1999) and transformed into wild type virulent M. marinum strain. Other promoters that can drive the expression of a downstream gene can substitute for PG13, both in normal growth or during infection of macrophages and in an animal susceptible to the infection. Transformed strains are organisms genetically and permanently modified with pG13A¹² vector and that have elevated levels of Δ¹²-desaturase mRNA that is translated in high level of the coded protein. Δ²-desaturase gene. The expression of Δ¹²-desaturase mRNA in transformed cells is shown in FIG. 22. Introduction and expression of Synechocystis PCC6803 Δ¹²-desaturase gene does not affect growth of the cells (FIG. 23). The introduction and over expression of Δ¹²-desaturase protein determines an increase in the Protein/Lipid ratio of M. marinum total membrane fractions transformed with pG13 Δ¹² containing Δ¹²-desaturase gene of Synechocystis PCC6803 under the control of PG13 promoter of about 40% (FIG. 24). FIG. 25 shows that survival of M. marinum transformed with pG13 Δ¹² containing the Δ¹² -desaturase gene of Synechocystis PCC6803 under the control of PG13 promoter is abolished while growth of control cells recovers as expected.

### EXAMPLE 3

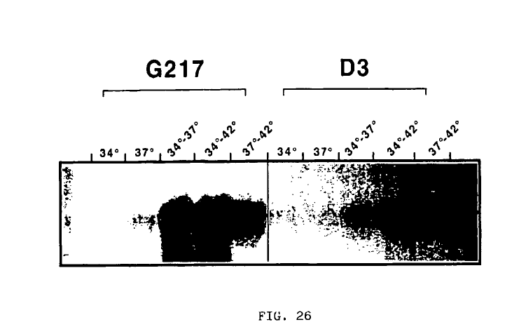

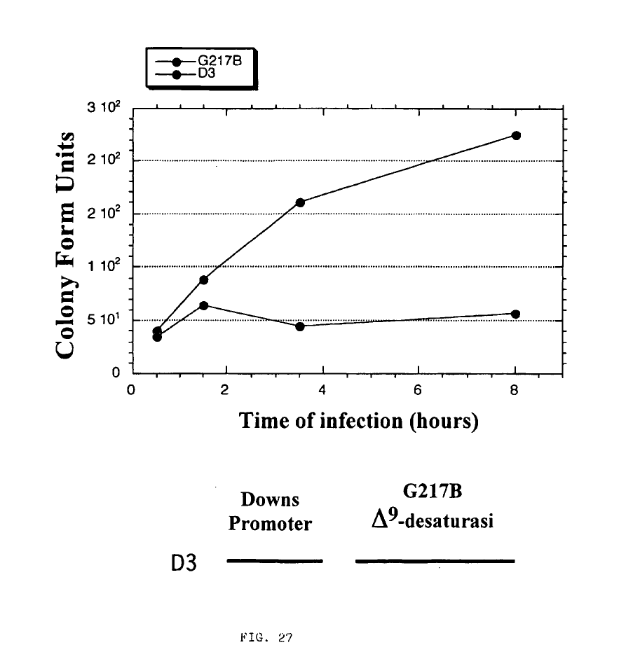

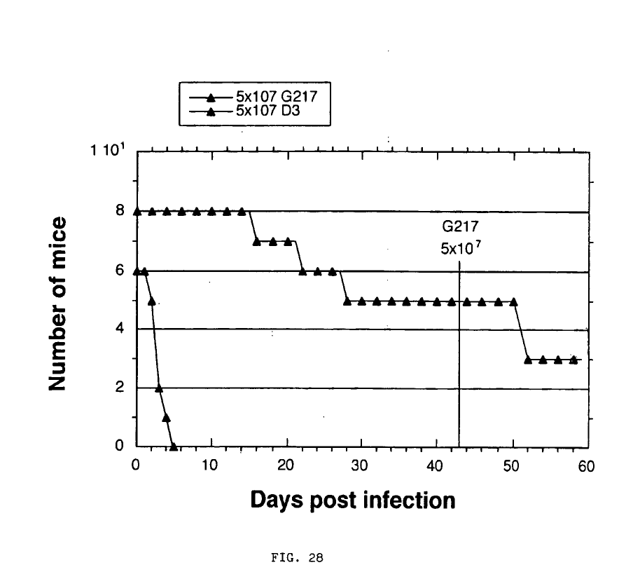

The entire Δ⁹-desaturase gene of virulent G217B strain of H. capsulatum has been cloned in pWU44 vector (Woods and Goldman, 1993) under the control of the up-regulated Δ⁹-desaturase promoter of the Downs strains of the fungus H. capsulatum (Gargano et al 1995) and used to transform the virulent G217B strain of H. capsulatum. Such modified strain has been denominated D3. Other promoters can substitute for such promoter capable of expressing the downstream gene either during normal growth or during infection of macrophages or of animals. The strains so transformed are the organisms genetically modified over expressing elevated level of Δ⁹-desaturase mRNA that is eventually translated in high level of the corresponding protein. Perturbation of MPS due to the over expression of Δ⁹-desaturase protein modifies the optimal temperature of expression of heat shock genes (e.g. hsp70) with a significant change in the pattern of accumulation of HSPs between 34° C. and 42° C. and in the hostile conditions present in the host's cells (e.g. macrophages and mammalian cells) (FIG. 26). These results consent to state that genetically obtained membrane perturbation by over expression of a desaturase gene or membrane proteins that locally or diffusely alter MPS, cause a significant change in the capacity of H. capsulatum (and that of other prokaryotic and eukaryotic intracellular and extracellular pathogens) to accumulate an appropriate amount of stress proteins. Membrane perturbation so obtained causes the complete and permanent loss of virulence of H. capsulatum that has been genetically transformed when it is used to infect macrophages obtained from tibiae of Balb CD-11 mice. FIG. 27 shows that D3 strain can not grow within murine macrophages. Balb CD-11 mice injected with yeasts of the high virulence G217B strain at the concentration of 5×10⁷/ml die within 5 days, while those injected with a similar amount of yeast cells of the genetically modified and attenuated D3 strain survive at least 45 days. Further, such mice, if challenged after the 45^{th} day with the virulent G217B strain show a survival rate of at least 60% (FIG. 28).

References

1. Buchmeier N A, et al. Science 248: 730-2, 1990.
2. Barker L P, Brooks D M, Small P L. Mol Microbiol. 29:1167-77,1998
3. Barker L P, Porcella S F, Wyatt R G, Small P L. FEMS Microbiol Lett. 175:79-85, 1999
4. Carratù, L., et al. Proc. Nat'l Acad. Sc. USA, 93: 3870-5, 1996
5. Cossins, A R (ed.) Temperature Adaptation of Biological Membranes, Portland Press, 1994
6. De Kruijff, B Nature 386:129-130,1997
7. Dunstan S A, et al. Inf Imm, 67: 5133-41, 1999
8. Feige U, et al. EXS 77: 359-73, 1996
9. Gargano S., et al. Lipids, 30: 899-906,1995
10. Groisman E A et al. Trends Microbiol 5:343-9, 1997
11. Groisman E A, et al. Trends Biochem Sci 15: 30-3,1990
12. Janoff A S, Haug & McGroarty E J Biochim. Biophys. Acta, 555: 56-66,1979
13. Horvath I, et al. Proc Natl Acad Sci USA. 95: 3513-8,1998
14. Lindgren S W, et al. Proc Natl Acad Sci USA 93: 4197-201, 1996
15. Lindquist S Annu Rev Biochem 55:1151-91,1986
16. Peterson G L Methods Enzymol. 91: 95-119,1983
17. Piper P W, et al. Cell Stress & Chaperones 2:12-24,1997
18. Sambrook J, et al. Molecular Cloning: A Laboratory Manual 1989, Cold Spring Harbor Laboratory Press, II Ed, Cold Spring Harbor, N.Y.
19. Singer M A, et al. Trends Biotechnol 16:460-8,1998
20. Slater S J, et al. J Biol Chem 269: 4866-4871, 1994
21. Suzuki I, et al. EMBO J 19:1327-34, 2000
22. Török Z, et al. Proc Natl Acad Sci USA. 98:3098-103, 2001
23. Tsuchido T, et al. J Gen Microbiol, 135: 1941-7, 1989
24. Van Eden & Young Eds, Stress proteins in Medicine, Dekker, N.Y., 1996
25. Vigh, L, et al. Trends Biochem Sci 23: 369-74,1998
26. Woods J P, et al. J Bacteriol; 175: 636-41, 1993

1-26. (canceled)

27. Method to transform pathogenic micro-organisms into non virulent ones comprising the step of modifying the physical and/or dynamic state of their biological membrane (MPS) by genetic modification of a gene whose product modulates the membrane saturated fatty acid/unsaturated fatty acid (SFA/UFA) or the protein/lipid ratios.

28. Method according to claim 27 wherein the modified physical and/or dynamic state of biological membranes alters the capacity of the pathogens to accumulate at the onset of infection an appropriate amount of stress proteins.

29. Method according to claim 27 wherein said genetic modification is an overexpression.

30. Method according to claim 27 wherein the genetic modification is obtained according to the following main steps: construction of a vector containing a gene under the control of a promoter, that regulates the expression of a gene whose protein product is able to modify the physical and/or dynamic state of the membranes of the microbial pathogens in which such vector is inserted; genetic transformation of pathogens with such a vector; expression of the protein product with such vector.

31. Method according to claim 30 wherein the gene whose protein product is able to modify the physical and/or dynamic state of the membranes of the microbial pathogens is selected in the group consisting of: desaturase genes, genes whose product is a membrane bound protein.

32. Method according to claim 31 wherein said desaturase gene is selected from: Δ¹²-desaturase gene, Δ⁹-desaturase gene, Δ⁶-desaturase gene, other desaturase genes, genes coding for integral membrane proteins.

33. Method according to claim 30 wherein said protein product is an enzyme that modifies the level of unsaturation of membrane fatty acids and phospholipids.

34. Method according to claim 30 wherein said membrane physical state modification consists in changes of membrane permeability.

35. Method according to claim 30 wherein said membrane physical state modification consists in changes of thermal phase transition profile of membrane.

36. Method according to claim 27 wherein the pathogenic micro-organisms are selected in the group of: strictly intracellular bacteria, facultative intracellular bacteria, fungi and parasites and non-intracellular pathogens.

37. Method according to claim 27 wherein the pathogenic microorganisms are selected in the group: Chlamydia species, such as pneumoniae and trachomatis, Coxiella burnetii, Ehrlichia chaffeensis, Rickettsiae; Legionella pneumophila, Mycobacteria, such as M. tuberculosis, M. marinum, M. leprae, Nocardia species (mycetoma), Bartonella species, Brucella species, Francisella tularensis, Listeria monocytogenes, Salmonella species, Shigella species; Borrelia burgdorferi (Lyme disease), Treponemapallidum, Campylobacter, Haemophilus influenzae Klebsiella pneumoniae, Leptospira interrogans, Neisseriae species, Staphylococci, Streptococcus pyrogenes, S. agalactiae, S. pneumoniae, Yersiniae, Bacillus anthracis; Aspergillus fumigatus, Candida species, Cryptococcus neoformans, Histoplasma capsulatum, Pneumocystis carinii; Entamoeba histolytica, Leishmania species, Plasmodium falciparum and vivax, Toxoplasma gondii, Trypanosoma cruzi.

38. Method for the production of attenuated non virulent pathogenic micro-organisms, selected among S. typhimurium, M marinum, and H. capsulatum, comprising the following steps: construction of a vector; transformation of the pathogen with such a vector that expresses, under the control of a promoter regulating the expression of a downstream gene during infection of the pathogen, one of the following genes: Cyanobacterium Synechocystis PCC6803 Δ¹²-desaturase gene or Δ⁹-desaturase of the virulent H. capsulatum G217B strain, S. cerevisiae or H. capsulatum Δ⁹-desaturase gene, or other desaturase genes of prokaryotic or eukaryotic organisms or other genes coding for integral membrane proteins that cause a perturbation of the physical and/or dynamic state of said biological membranes; over-expression of the inserted gene.

39. Method according to claim 38 wherein the promoter is an up-regulated promoter of the Downs strain.

40. Salmonella obtained with a method according to claim 38 characterized by a protein/lipid ratio of isolated outer membrane that is about 100 in the virulent strain and 170 in the genetically modified strain.

41. M. marinum obtained with method according claim 38 characterized by protein/lipid ratio of the isolated outer membrane that is increased 40% in the genetically modified strain.

42. Pathogenic micro-organisms obtained with the method according to claim 27 and characterized for being attenuated in their mechanism of virulence but immuno-competent.

43. Modified micro-organisms obtained with the method according to claim 27 to be used in medical applications.

44. Modified micro-organisms obtained with the method according to claim 27 for the manufacture of a vaccine.

45. Use of modified micro-organisms according to claim 43 to produce vaccines.

46. Vaccines comprising as active ingredient an effective amount of modified microorganisms according to claim 44 in combination with suitable excipients and additives.

47. Vaccines according to claim 46 in which the microorganisms are selected in the group comprising: Chiamydia species, such as pneumoniae e trachomatis, Coxiella burnetii, Ehrlichia chaffeensis, Rickettsiae; Legionella pneumophila, Mycobacteria, such as M. tuberculosis, M. marinum, M. leprae, Nocardia species (mycetoma), Bartonella species, Brucella species, Francisella tularensis, Listeria monocytogenes, Salmonella species, Shigella species; Borrelia burgdorferi (Lyme disease), Treponema pallidum, Campylobacter, Haemophilus influenzae Klebsiella pneumoniae, Leptospira interrogans, Neisseriae species, Staphylococci, Streptococcus pyrogenes, S. agalactiae, S. pneumoniae, Yersiniae, Bacillus anthracis; Aspergillus fumigatus, Candida species, Cryptococcus neoformans, Histoplasma capsulatum, Pneumocystis carinii; Entamoeba histolytica, Leishmania species, Plasmodium falciparum, vivax, Toxoplasma gondii, Trypanosoma cruzi, and relative combinations.

48. Vaccines according to claim 46 formulated to be administered intradermically, intramuscularly, intravenously, in the mucosa, nasally, vaginally, orally and rectally.


SEQUENCE LISTING (4 sequences)

SEQ ID NO: 1 - DNA, 1489 bp, Synechocystis PCC6803
  CDS (167)..(1219)
aattcagaagcaattcggttcctggtcaatggcaacgtgttataaaaagaaaagtttgtttacctgagtattaattcctaggcacggcaaaccttggccgctttatagcccatgaatccataaacaaaatctgtccgaccttccatttggagataaacctttataaatgactgccacgattcccccgttgacaccaacggtaacgcccagcaatcccgatcgcccgattgcggatctcaaactacaagacatcattaaaaccctgcccaaggaatgcttcgagaaaaaagcgagcaaagcctgggcttctgttttgattaccctaggggcgatcgccgtgggctatttgggcattatttatctgccctggtactgcttgcccattacctggatctggacagggacagccttaacgggggccttcgttgtcggccatgactgtggccatcgctcctttgctaaaaaacgctgggtcaatgatttagtgggacatatcgcttttgctcccctcatctaccctttccatagctggcgcctactccacgaccaccatcacctccacaccaacaaaattgaggttgataacgcctgggatccctggagtgtggaagctttccaagccagcccggcgatcgtccggcttttttatcgggccatccggggtcccttctggtggactggttccattttccattggagcttaatgcacttcaaactttccaactttgcccaaagggaccgcaataaagtcaaattatccattgccgttgtcttcctgtttgcggcgatcgcctttcctgccctaattatcaccacaggggtgtggggtttcgtcaaattttggctaatgccctggttggtgtatcacttttggatgagcacttttaccattgtgcaccacaccattcccgaaattcgtttccgtcccgccgccgattggagtgccgccgaagcccagttaaatggtactgttcactgcgattatccccgttgggtggaagtgctctgccatgacatcaacgtccatattccccaccacctctccgttgccatcccttcctataacctacgactagcccacggaagtttaaaagaaaactggggaccttttctttacgagcgcacctttaactggcaattaatgcaacaaattagtgggcaatgtcatttatatgaccccgaacatggctaccgcaccttcggctccctgaaaaaagtttaatactgggacaactagtaatttttgacccatgattggtcagtaattaactttgactgatccccagggagagaaataccagatcacaaattaactatcttggaatgcggccatcgagctgtattccttttttcttttcttggtgaggaaaaaaactttctaagtgggcagatgggagcggttcagactagaaagatccactcggccaaaatgaatgctaaacgcaacctgcatattctccaaggttttcactagccaaggtaccccttc

SEQ ID NO: 2 - protein, 351 aa, Synechocystis PCC6803
MTATIPPLTPTVTPSNPDRPIADLKLQDIIKTLPKECFEKKASKAWASVLITLGAIAVGYLGIIYLPWYCLPITWIWTGTALTGAFVVGHDCGHRSFAKKRWVNDLVGHIAFAPLIYPFHSWRLLHDHHHLHTNKIEVDNAWDPWSVEAFQASPAIVRLFYRAIRGPFWWTGSIFHWSLMHFKLSNFAQRDRNKVKLSIAVVFLFAAIAFPALIITTGVWGFVKFWLMPWLVYHFWMSTFTIVHHTIPEIRFRPAADWSAAEAQLNGTVHCDYPRWVEVLCHDINVHIPHHLSVAIPSYNLRLAHGSLKENWGPFLYERTFNWQLMQQISGQCHLYDPEHGYRTFGSLKKV

SEQ ID NO: 3 - DNA, 24 bp, Artificial Sequence
  oligonucleotide
caaattatgaccctgttgatcagt

SEQ ID NO: 4 - DNA, 25 bp, artificial sequence
  oligonucleotide
cccccccatatgactgccacgattc